(19) United States
(12) Patent Application Publication
(10) Pub. No.: US 2025/0339229 A1
(43) Pub. Date: Nov. 6, 2025

(54) LINE MANAGEMENT APPARATUS

(71) Applicant: James Cameron Dodds, Greenville, SC (US)

(72) Inventors: James Cameron Dodds, Greenville, SC (US); Lisa Fitzgerald, Leesburg, FL (US)

(21) Appl. No.: 19/264,365

(22) Filed: Jul. 9, 2025

Related U.S. Application Data
(63) Continuation-in-part of application No. 18/941,415, filed on Nov. 8, 2024, now Pat. No. 12,390,297.
Application No. 18/941,415 is a continuation of application No. 18/409,073, filed on Jan. 10, 2024, now Pat. No. 12,138,086.
Application No. 18/409,073 is a continuation of application No. 18/221,570, filed on Jul. 13, 2023, now Pat. No. 11,877,872.
(60) Provisional application No. 63/462,063, filed on Apr. 26, 2023.

(51) Int. Cl. A61B 50/20

(52) U.S. Cl. CPC A61B 50/20

(57) ABSTRACT

A spool assembly includes a spool; a frame, a biasing element applying torque against rotation of the spool in one direction, and a flexible line variably wound on the spool. When the line is extended by unwinding from the spool, the spool is rotated in the one direction against the torque. A stop device coupled to the spool selectively prevents rewinding. A release device is coupled to the stop device and upon user action thereon, the release device releases the stop device to allow the spool to rotate opposite the one direction by the torque provided by the biasing element thereby selectively permitting retraction of the flexible line by rewinding at least a portion thereof onto the spool. A rotary damper operatively coupled to the spool to damp rotation of the spool during rewinding.

### CROSS-REFERENCE TO RELATED APPLICATION

This application is a continuation-in-part (CIP) of U.S. non-provisional patent application Ser. No. 18/941,415, “Vital Signs Monitor Cable Retraction System,” filed on Nov. 8, 2024, which is a continuation of U.S. non-provisional patent application Ser. No. 18/409,073, which is a continuation of U.S. non-provisional patent application Ser. No. 18/221,570 titled “Vital Signs Monitor Cable Retraction System,” filed on Jul. 13, 2023 (now U.S. Pat. No. 11,877,872), which claims the benefit of priority of U.S. provisional patent application No. 63/462,063 titled “Vital Signs Monitor Cable Retraction System,” filed on Apr. 26, 2023. Each above-referenced application is incorporated herein in its entirety by this reference.

### TECHNICAL FIELD

The present invention relates generally to the field of spring-driven retraction spools for cables, wiring and tubes, and more particularly, to multi-line spring-driven retraction devices for medical devices.

### BACKGROUND

The vital sign monitor is used by hospitals, clinics, and ambulances to monitor a patient's vital signs that typically include measurement of blood pressure, oxygen saturation, pulse, EKG, and temperature. Each of these measurements require a sensor connected to a cable or tube, from the sensor to the monitor, where results can be displayed on a screen. In a typical setting, these cables and tubes can become tangled and, left un-stored, can be damaged. The sensors can fall to the floor where damage may occur. This condition places a burden on the clinical staff that is under time constraints to monitor vital signs of a patient and is then required to untangle cords or replace sensors. Historically these sensors and cables were draped over the monitor or placed in a basket below the monitor. In both cases this would not guarantee that tangles may occur and un-secured sensors may be damaged.

Dentists and dental hygienists also use hand-held devices that are powered by, dispense, and collect fluids. A patient receiving dental care in many cases is awake and aware, having entered a care-treatment room on foot and likely the leave the same way. Tidy equipment would serve both sanitary and aesthetics in medical facilities, dental care facilities, and elsewhere.

A need exists to provide better handling of the cables, tubes and sensors that solves the above-mentioned problems.

### SUMMARY

This summary is provided to briefly introduce concepts that are further described in the following detailed descriptions. This summary is not intended to identify key features or essential features of the claimed subject matter, nor is it to be construed as limiting the scope of the claimed subject matter.

A spool assembly, according to at least one embodiment, includes: a spool having a rotation axis; a fixed frame on which the spool is mounted; a biasing element operatively coupled to the spool to apply a torque against rotation of the spool in a first rotational direction around the rotation axis relative to the fixed frame; a flexible line mounted on the spool in a variably wound condition such that, when the flexible line is extended by unwinding from the spool, the spool is rotated in the first rotational direction against the torque provided by the biasing element; a stop device coupled to the spool selectively preventing rotation of the spool in a second rotational direction opposite the first rotational direction thereby selectively preventing, by opposing at least the torque provided by the biasing element, retraction of the flexible line by winding onto the spool; a release device operatively coupled to the stop device such that, upon action on the release device, the release device selectively releases the stop device to allow the spool to rotate in the second rotational direction by the torque provided by the biasing element thereby selectively permitting retraction of the flexible line by rewinding at least a portion thereof onto the spool; and a rotary damper operatively coupled to the spool to damp rotation of the spool at least in the second rotational direction.

A hub may be connected to the spool to rotate therewith, the hub comprising a smooth cylindrical axle, and the stop device may include a clutch element at least partially surrounding a smooth cylindrical clutch engagement portion of the axle, the clutch element having an engaged condition and a disengaged condition. In the engaged condition, the clutch element selectively prevents rotation of the axle and spool therewith in the second rotational direction by frictional engagement with the smooth cylindrical clutch engagement portion of the axle.

The hub may include a spur gear engaged by the rotary damper.

The clutch element may be a torsion spring mounted around the smooth cylindrical clutch engagement portion of the axle, and in the engaged condition of the clutch element the torsion spring prevents rotation of the axle and spool therewith in the second rotational direction by frictional engagement with the clutch engagement portion of the axle.

The release device may be operatively coupled to the torsion spring to vary a wound condition of the torsion spring around and upon the clutch engagement portion of the axle.

A first end of the torsion spring may be operatively coupled to the release device; and a second end of the torsion spring opposite the first end may be irrotationally engaged with the fixed frame.

The release device may include a clutch release cap engaged with the first end of the torsion spring such that upon a pivoting of the clutch release cap around the rotation axis, the wound condition of the torsion spring around and upon the clutch engagement portion of the axle is loosened.

The release device may include a plunger mounted on the fixed frame and a link member operatively coupling the plunger to the clutch release cap such that linear movement of the plunger causes pivoting of the clutch release cap around the rotation axis.

The clutch release cap may include a flange having multiple spaced engagement features for selection of use by which to engage the link member and clutch-release cap for adjustment of the clutch element.

The biasing element may include a recoil spring within the spool, and a spring stay may be connected irrotationally to the fixed frame. An interior end portion of the recoil spring may engage the spring stay and be prevented from rotating relative to the fixed frame by the spring stay. An exterior hooked end portion of the recoil spring may engage an interior feature of the spool such that the recoil spring applies said torque against rotation of the spool in the first rotational direction.

The recoil spring may be configured such that, when the flexible line is extended by unwinding from the spool, the spool is rotated in the first rotational direction and an increasing tightly wound interior portion of the recoil spring is accumulated around the spring stay, tightening the recoil spring.

The fixed frame may include a mounting plate, a first stanchion mounted on the mounting plate, a second stanchion mounted on the mounting plate and spaced from the first stanchion; and a cover plate mounted on the first stanchion and second stanchion and spaced from the mounting plate. The spool may be mounted on the frame between the first stanchion and second stanchion, and between the mounting plate and cover plate.

The spool may include: a cylinder defining an engagement surface on which the flexible line is mounted on the spool in the variably wound condition; a first flange connected to the cylinder and extending radially outward from the cylinder for maintaining the flexible line on the spool in the variably wound condition; and a second flange connected to the cylinder, spaced from the first flange by the engagement surface, and extending radially outward from the cylinder for maintaining the flexible line on the spool in the variably wound condition.

The spool may have a slot through the cylinder and through which a terminal end of a flexible line passes through the cylinder.

The flexible line can be configured to carry, convey, contain, collect and/or dispense at least one of a signal and a flowable substance.

A cover may be connected directly or indirectly to the fixed frame and at least partially defining an enclosure around the spool assembly.

The above summary is to be understood as cumulative and inclusive. The above and below described features are to be understood as combined in whole or in part in various embodiments whether expressly described herein or implied by at least this reference. For brevity, not all features are expressly described and illustrated as combined with all other features. No combination of features shall be deemed unsupported for merely not appearing expressly in the drawings and descriptions.

### BRIEF DESCRIPTION OF THE DRAWINGS

The previous summary and the following detailed descriptions are to be read in view of the drawings, which illustrate some, but not all, embodiments and features as briefly described below. The summary and detailed descriptions, however, are not limited to only those embodiments and features explicitly illustrated.

FIG. 1 is a perspective view of the full assembly of a cable retract system according to at least one embodiment, shown with attached medical devices as extending forward and a vital signs monitor rearward.

FIG. 2 is a perspective view of the cable retract system of FIG. 1 with the cover removed.

FIG. 3 is an enlarged detail view of a portion FIG. 2.

FIG. 4 is a front view of the cable retract system of FIG. 1.

FIG. 5 is a side view of the cable retract system of FIG. 1.

FIG. 6 is a bottom view of the cable retract system of FIG. 1.

FIG. 7 is a back view of the cable retract system of FIG. 1.

FIG. 8 is a top view of the cable retract system of FIG. 1.

FIG. 9 is a rear view of the cable retract system of FIG. 1 with the cover removed.

FIG. 10 is a section view taken along the line 10-10 in FIG. 9.

FIG. 11 is a section view taken along the line 11-11 in FIG. 9.

FIG. 12 is a section view taken along the line 12-12 in FIG. 9.

FIG. 13 is a section view taken along the line 13-13 in FIG. 9.

FIG. 14 is a section view taken along the line 14-14 in FIG. 9.

FIG. 15 is a section view taken along the line 15-15 in FIG. 9.

FIG. 16 is an exploded perspective view of the cable retract system of FIG. 1.

FIG. 17 is an exploded perspective view of a spool assembly, according to at least one embodiment.

FIG. 18 is an exploded view of the spool assembly of FIG. 17 from another perspective.

FIG. 19 is an exploded perspective view of a spool assembly having a tube and an air fitting.

FIG. 20 is another front view of the cable retract system of FIG. 1.

FIG. 21 is a section view taken along the line 21-21 in FIG. 20.

FIG. 22 is a perspective view of a line management apparatus according to another embodiment.

FIG. 23 is a side view of the line management apparatus of FIG. 22.

FIG. 24 is a bottom base view of the line management apparatus of FIG. 22.

FIG. 25 is a perspective view of a mounting bracket for optional use with the line management apparatus of FIG. 22.

FIG. 26 is a perspective view taken as in FIG. 22 of the line management apparatus with the cover removed for illustration of components.

FIG. 27 is a side view taken as in FIG. 23 of the line management apparatus with the cover removed for illustration of components.

FIG. 28 is a back-end perspective view of the line management apparatus of FIG. 22 without the cover.

FIG. 29 is a perspective view of the base plate with a single mounted spool assembly of the line management apparatus of FIG. 22.

FIG. 30 is a front elevation view of a single spool assembly according to at least one embodiment.

FIG. 31 is back elevation view opposite FIG. 30 of the spool assembly thereof.

FIG. 32 is a first side view of the spool assembly of FIG. 30.

FIG. 33A is a second side view opposite FIG. 32 of the spool assembly thereof.

FIG. 33B is a downward viewing perspective view of a fixed frame of the spool assembly of FIG. 33A.

FIG. 34 is an exploded side view of the spool assembly taken from the perspective of FIG. 32.

FIG. 35 is an exploded perspective view of the spool assembly of FIG. 22.

FIG. 36 is a frontal exploded view of the spool assembly of FIG. 22.

FIG. 37 is an exploded perspective view of front-end components of the spool assembly of FIG. 22.

FIG. 38 is a perspective view of the clutch release cap of the spool assembly of FIG. 22.

FIG. 39 is a perspective view of the clutch element of the spool assembly of FIG. 22.

FIG. 40 is an exploded perspective view of back-end components of the spool assembly of FIG. 22.

FIG. 41 is another exploded perspective view of back-end components of the spool assembly of FIG. 22.

FIG. 42 is a perspective view of the recoil spring of the spool assembly of FIG. 22.

FIG. 43 is a back-end view of the spool of the spool assembly of FIG. 22.

FIG. 44 is a front-end perspective view of the spool of the spool assembly of FIG. 22.

FIG. 45 is a perspective view of the front end of the spool of FIGS. 43 and 44, shown partially assembled with a rotary fluid connection device.

FIG. 46 is a perspective view of the rotary fluid connection device of FIG. 45.

FIG. 47 is an exploded view of the spool and rotary fluid connection device of FIGS. 45.

FIG. 48 is a perspective view of the front stanchion, hub, and a rotary electrical connection device for use with the spool assembly of FIG. 22.

### DETAILED DESCRIPTIONS

The present invention will now be described more fully, hereinafter with reference to the accompanying drawings in which exemplary embodiments of the invention are shown. However, the invention may be embodied in many different forms and should not be construed as limited to the representative embodiments set forth herein. The exemplary embodiments are provided so that this disclosure will be both thorough and complete and will fully convey the scope of the invention and enable one of ordinary skill in the art to make, use and practice the invention. Like reference numbers refer to like elements throughout the various drawings.

The present invention will now be described more fully hereinafter with reference to the accompanying drawings in which exemplary embodiments of the invention are shown. However, the invention may be embodied in many different forms, and should not be construed as limited to the representative embodiments set forth herein. The exemplary embodiments are provided so that this disclosure will be both thorough and complete and will fully convey the scope of the invention and enable one of ordinary skill in the art to make, use and practice the invention.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings. However, for each drawing, at least one embodiment is made according to the apparent relative scale of the drawing.

Like reference numbers used throughout the drawings depict like or similar elements. Unless described or implied as exclusive alternatives, features throughout the drawings and descriptions should be taken as cumulative, such that features expressly associated with some particular embodiments can be combined with other embodiments.

Unless defined otherwise, technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a,” “an,” and “the” refer to “one or more” when used in the subject specification, including the claims.

These descriptions and the referenced drawings specifically disclose a Vital Signs Monitor Cable Retraction System that provides a means to store the cables and tubes on spools that can be pulled out for use and then retracted back on the spools, by means of rotational springs, when not needed. Uses other than that described with reference to vital signs monitoring are within the scope of these descriptions and drawings.

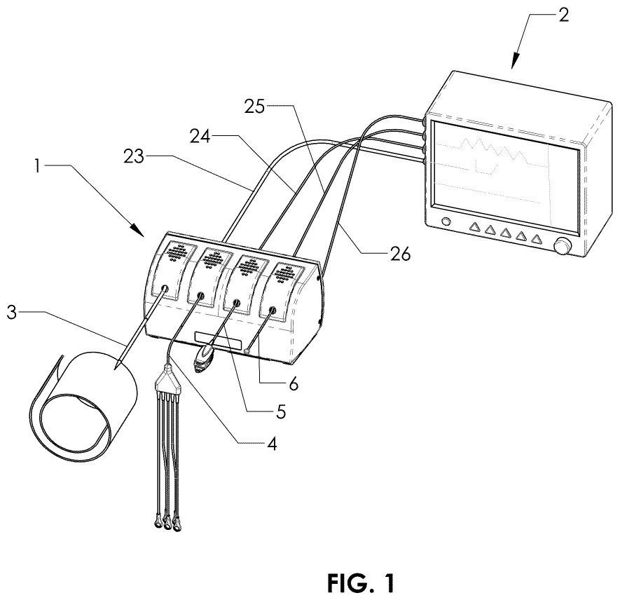

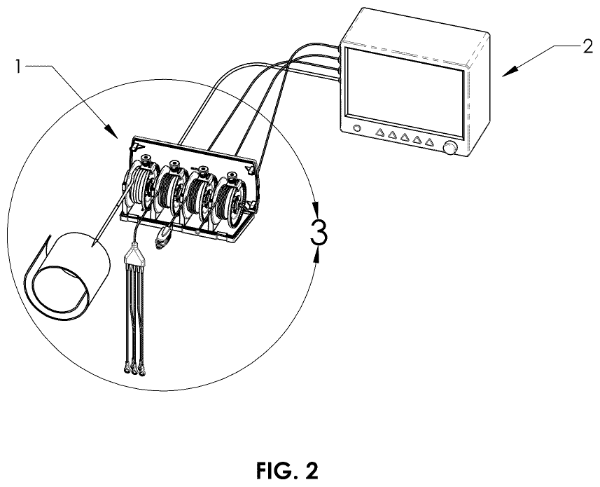

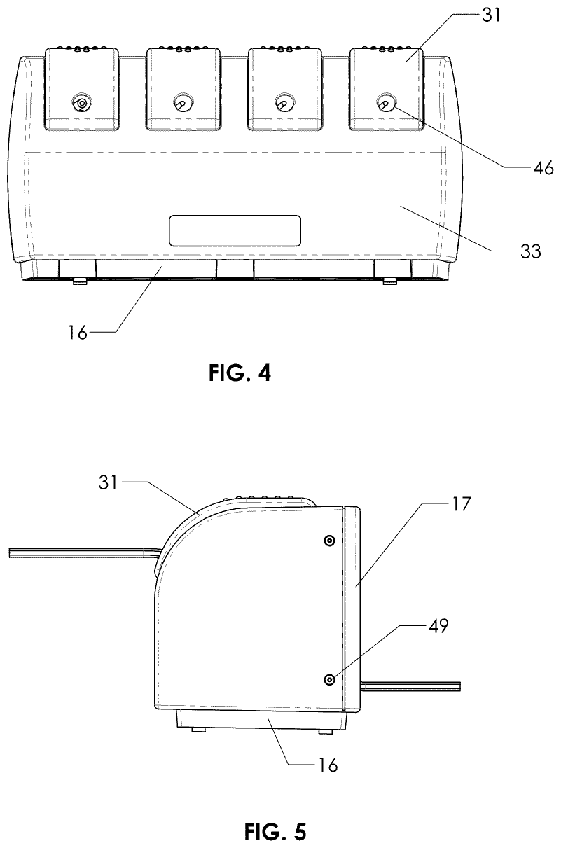

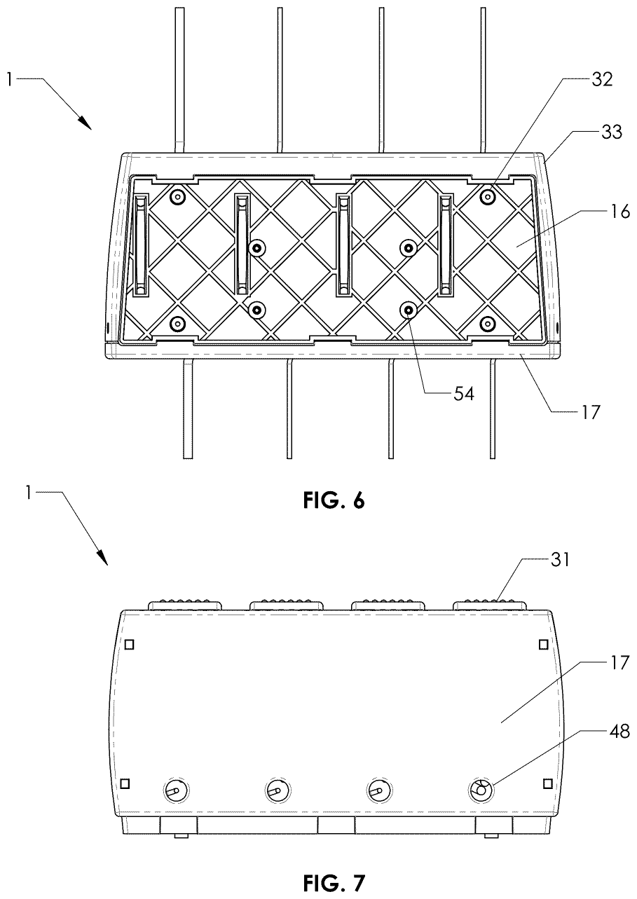

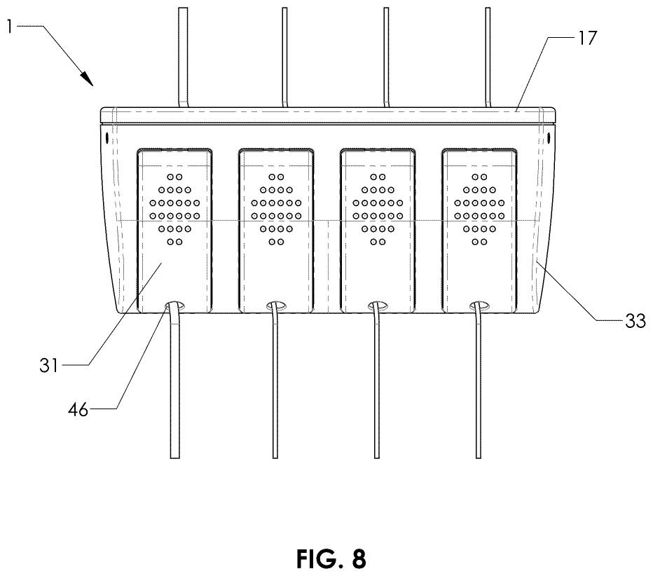

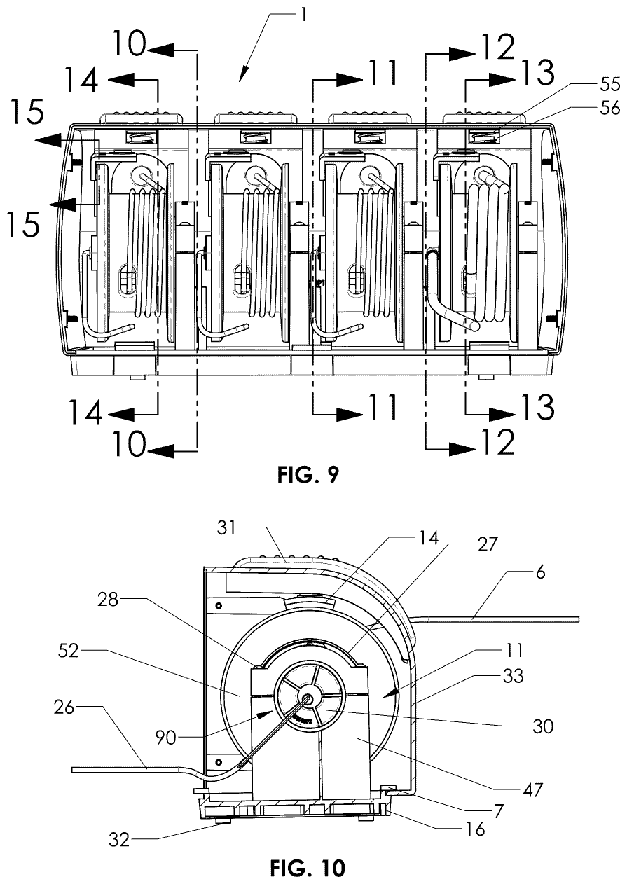

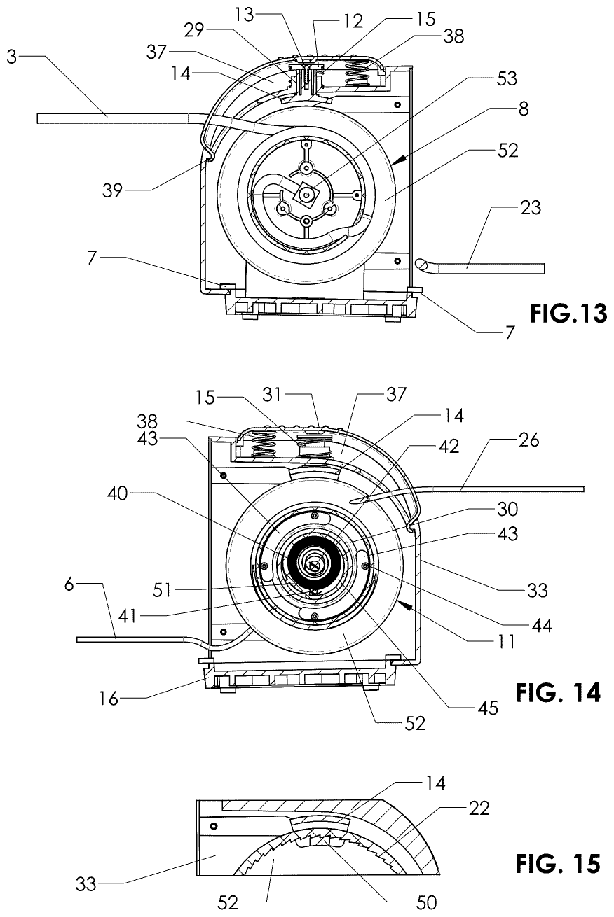

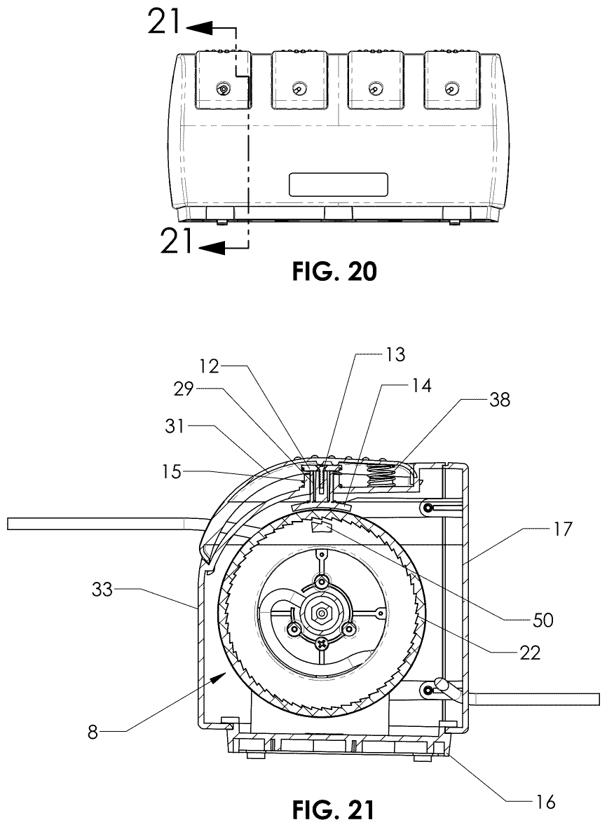

The Vital Sign Monitor Cable Retract System, in the illustrated and/or described embodiment(s) of FIGS. 1-21, includes spool assemblies in an enclosure that allows cables and tubes to be wound fully retracted into the enclosure. This organizes the cables or tubing, prevents tangles and provides protection for the sensor assemblies by being hung high off the floor. In this embodiment, there are four spools, one for blood pressure, oxygen saturation/pulse, EKG, and temperature. In other embodiments, the number of spool assemblies can vary as the requirement for sensors dictate.

Each spool assembly consists of a spool that is rotationally biased by a spring in a partially wound condition thereby retracting the cable or tubing on the spool. As the cable or tubing is extended, further biasing the spring, a ratcheting stop device prevents retraction of the cable or tubing. A lever for each spool assembly, attached to the enclosure, releases the ratchet to allow the spool to rewind the sensor cable or tubing. To prevent twisting of the cable, a slip-ring assembly is mounted to each cable spool coincident to the center axis. To prevent twisting of the tubing, an air rotational fitting is mounted to the tubing spool coincidental to the center axis. The output of the slipring and air rotational fitting transverses through apertures in the rear panel of the enclosure and plugs into the vital signs monitor. A screw boss interface on the bottom of the enclosure provides a mount point for a pole or wall bracket (not shown) to be attached.

Additional features, aspects and advantages of the invention will be set forth in the detailed description which follows, and in part will be readily apparent to those skilled in the art from that description or recognized by practicing the invention as described herein. It should be understood that both the foregoing general description and the following detailed description present various embodiments of the invention and are intended to provide an overview or framework for understanding the nature and character of the invention as it is claimed. The accompanying drawings are included to provide a further understanding of the invention and are incorporated in and constitute a part of this specification.

Referring to FIGS. 1, 2, 3, 4, 5, 6, 7, 8, 9, 10, 11 and 16, the Vital Signs Monitor Cable Retraction System includes, in the illustrated embodiment, a cable retraction device 1 containing four spool assemblies, the blood pressure hose spool assembly 8, the EKG cable spool assembly 9, the oxygen/pulse cable spool assembly 10 and the temperature cable spool assembly 11. The cable retraction device 1 enclosure is comprised of top cover 33, rear panel 17 and bottom mainframe 16. Rear panel 17 includes a plurality of apertures 48 for cables and tubing to exit the device. Bottom mainframe 16 is captured between the rear panel 17 and the top cover 33 by a plurality of tabs 7. Rear panel 17 and the top cover 33 are secured together by rear panel attachment screws 49. Rubber feet 32 provides table-top grip. Mounting bracket screw boss 54, optionally an integral feature of bottom mainframe 16, provides a mounting point for a pole or wall bracket (not shown) to be attached. Wire retainer 34, optionally an integral feature of bottom mainframe 16, provides a channel to secure cables to avoid chaffing on the rotating spools.

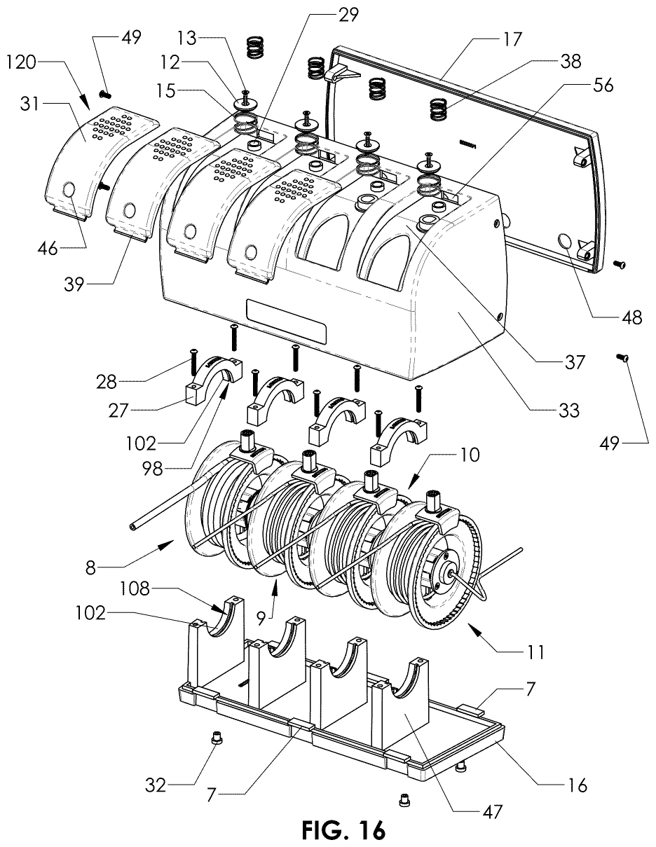

Referring to FIGS. 10 and 16, the blood pressure hose spool assembly 8, the EKG cable spool assembly 9, the oxygen/pulse cable spool assembly 10 and the temperature cable spool assembly 11 are each constrained to the bottom mainframe 16 by a respective axle race clamp 27 capturing an axle race 30 on each of the spool assemblies into a plurality of mainframe cradles 47, optionally an integral feature of bottom mainframe 16, by means of spool axle clamp screw 28. This clamp fixes the axle race 30, thereby all spool assemblies, in all axis and rotation.

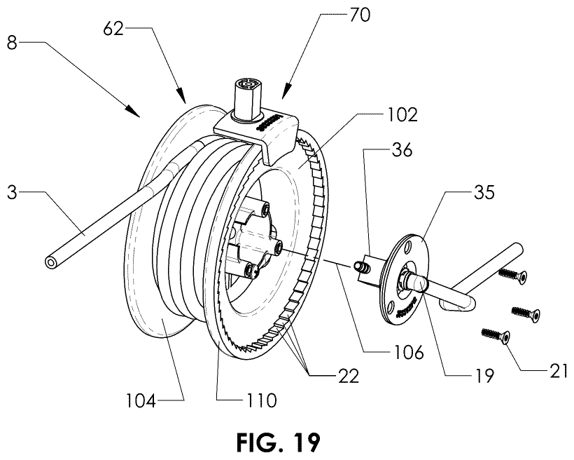

Referring to FIGS. 14, 17, 18 and 19, cable retraction device 1 contains a plurality of spool assemblies, each similarly consisting of a spool 52, spool axle 51, optionally an integral part of spool 52, a constant force rotary spring 40 and an axle race 30. As the spool rotates due to tube or cable extraction, the constant force rotary spring 40, located in spring well 45, optionally an integral part of spool 52, creates a rotary spring bias between the spool 52 and the axle race 30 required to retract the cable or tube. Rotary spring retainer screw 41 secures the constant force rotary spring 40 to spool 52. The wound end of the spring is engaged and wound by spring winder 42 constrained in axle race 30 by spring winder screw 53. Axle race 30 is retained to the spool 52 by two axle race retainers 43 constrained by axle race retainer screws 44. A slip-ring assembly 18 is constrained to the center axis of spool 52 by slip-ring retainer screws 20. Referring to FIG. 19, in the case of the blood pressure hose spool assembly 8, an air fitting 36 and air rotary joint fitting 19 are constrained to the center axis of the spool 52 by the air rotary joint plate 35 and air rotary joint plate retainer screws 21.

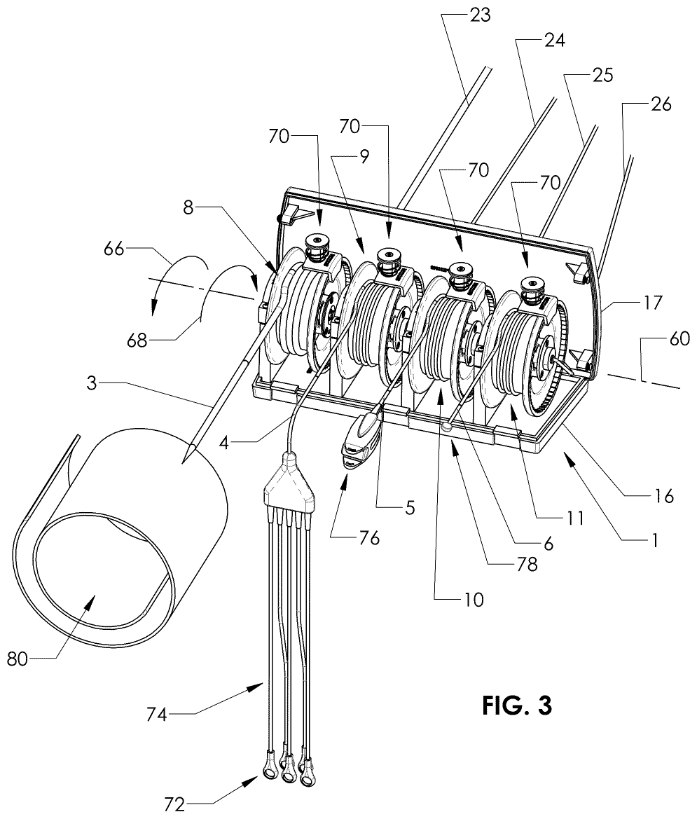

Referring to FIGS. 3, 9, 11, 12, 13, 14, 15, 20 and 21, the blood pressure hose spool assembly 8, the EKG cable spool assembly 9, the oxygen/pulse cable spool assembly 10 and the temperature cable spool assembly 11 are all locked in the fully extended position by an identical mechanism consisting of a spool release plunger 14 with integral ratchet teeth 50. Ratchet teeth 50 engage with spool locking teeth 22, optionally an integral feature of spool 52. Ratchet teeth 50 are biased into locking teeth 22 by spool release spring 15 biased between the top cover 33 and the spool release spring retainer 12. Spool release spring retainer 12 is connected to the spool release plunger 14 by means of a spool release spring retainer screw 13. Spring bias is translated by the spool release plunger 14 through the release plunger aperture 29, optionally an integral feature of top cover 33, and onto the locking ratchet teeth 50. As the cable or tube is extracted, the bias is overcome, and the spools are free to rotate. The geometry of the tooth disables the spool from rotating in the opposite direction. To free the spool and allow retraction of the cables or tubing, the release plunger 14 is depressed by means of release lever 31 thereby disengaging ratchet teeth 50 from the locking teeth 22 on spool 52. Release lever 31 is captured by the release lever pivot 39, optionally an integral feature of top cover 33, on the distal end and snapped into the release lever tab aperture 56, optionally an integral feature of top cover 33, by means of release lever tab 55, optionally an integral feature of release lever 31, on the proximal end. The release lever 31 is biased by release lever spring 38 and top cover 33, fully unengaged. To engage release lever 31, thereby depressing release plunger 14 to release the spool, the release lever 31 is pushed into the release lever well 37, optionally an integral feature of top cover 33. Spool release lever aperture 46, integral feature of release lever 31, allows entry of the cables or tubes onto the spools.

Referring to FIGS. 1, 2, 7, 12 and 19, the blood pressure cuff tube 3 is wound around blood pressure hose spool assembly 8 though air fitting 36 and air rotary joint fitting 19. The air rotary joint fitting 19 prevents the blood pressure cuff tube 3 from twisting as the blood pressure hose spool assembly 8 rotates. The blood pressure cuff tube 3 is connected to the vital signs monitor 2 by means of the blood pressure monitor tube 23 that connects to the air rotary joint fitting 19, then passing through the corresponding rear panel aperture 48 on rear panel 17 and connecting to the vital signs monitor 2.

Referring to FIGS. 1, 2, 3, 7, 16, 17 and 18, the EKG harness cable 4, oxygen/pulse sensor cable 5 and temperature sensor cable 6 is wound around the corresponding EKG cable spool assembly 9 oxygen/pulse cable spool assembly 10 and temperature cable spool assembly 11 are wound around their corresponding spool assemblies. Each cable connects to an electrically conducting slip ring assembly 18. The slip ring assemblies 18 prevents the cables from twisting as the spool assemblies rotate. The cables are connected to the vital signs monitor 2 by means of the EKG monitor cable 24, the oxygen/pulse monitor cable 25 and the temperature monitor cable 26 that connects to the corresponding slip-ring assemblies 18. The cables pass through the corresponding rear panel apertures 48 on rear panel 17 and then connected to the vital signs monitor 2.

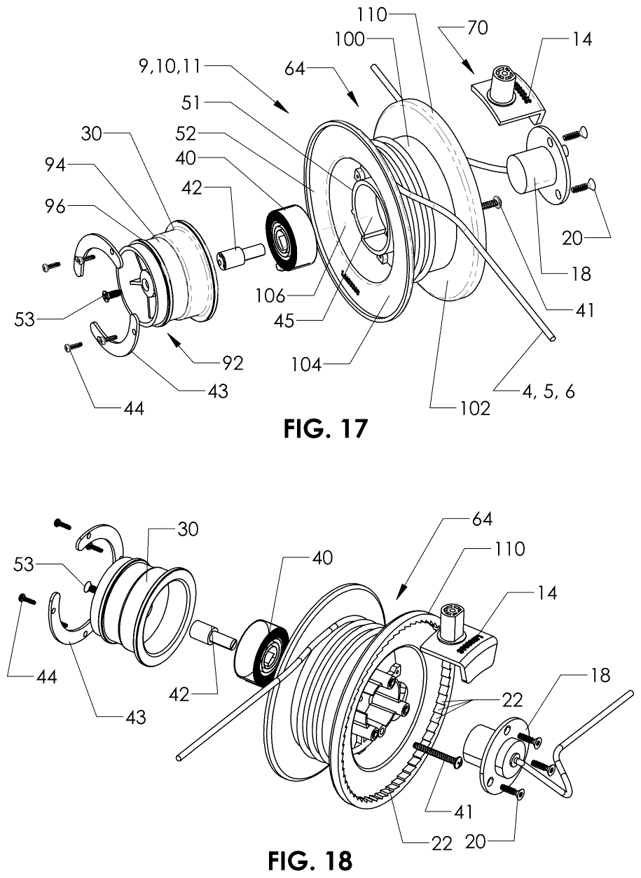

In accordance with the preceding descriptions, the cable retraction device 1 defines least one embodiment of a spooled line handling system having multiple spool assemblies 8, 9, 10, 11 spaced along a common axis 60 (FIG. 3), and a mainframe 16 at least on which the spool assemblies are mounted. Each one of the multiple spool assemblies 8, 9, 10, 11 includes a respective flexible line, with reference at least to the illustrated and non-limiting examples: blood pressure cuff tube 3; EKG harness cable 4; oxygen/pulse sensor cable 5; and temperature sensor cable 6. Each one of the multiple spool assemblies 8, 9, 10, 11 further includes a spool rotationally biased by a spring torque, with reference to the illustrated spool 62 (FIG. 19) with respect to the spool assembly 8, and with reference to the illustrated spool 64 (FIG. 17) with respect to the spool assemblies 9, 10, and 11.

The respective flexible line 3, 4, 5, 6 is mounted on each spool 62, 64, 64, 64 in a variably wound condition such that, as the respective flexible line is extended by unwinding from the spool, the spool is rotated in a first rotational direction 66 (FIG. 3) against the spring torque around the common axis 60 of the multiple spool assemblies. A respective stop device 70 is coupled to each spool selectively preventing rotation of the spool in a second rotational direction 68 (FIG. 3) opposite the first rotational direction 66 thereby selectively preventing, by opposing the spring torque, retraction of the respective flexible line by winding onto the spool.

Each one of the multiple spool assemblies 8, 9, 10, 11 includes a respective release device, with reference at least to the illustrated release levers 31, movably attached directly or indirectly to the mainframe and operatively coupled to the stop device 70. Upon user action on the release device, the release device selectively releases the stop device 70 to allow the spool to rotate in the second rotational direction 68 by the spring torque thereby selectively permitting retraction of the respective flexible line by rewinding at least a portion thereof onto the spool.

In use, a user such as a medical professional manually pulls on any selected flexible line to extend the line. When the extended portion of the flexible line is to be returned to its spool assembly, the user can press on the release lever 31, defining user action thereon, permitting retraction of the respective flexible line.

In the illustrated embodiment of the spooled line handling system, the respective flexible line of at least one of said multiple spool assemblies comprises a cable comprising at least one electrically conducting wire, with reference at least to the respective flexible lines 4, 5, 6 of the spool assemblies 9, 10, and 11. As represented in FIG. 3, the system can include or can be coupled to at least one sensor connected to the at least one electrically conducting wire. In the illustrated example (FIG. 3), each flexible line 4, 5, 6 includes an electrically conducting signal wire for a respective sensor. In particular: the flexible line 4 is illustrated as an EKG harness cable from which multiple EKG pad sensors 72 extend on respective cables 74 (FIG. 3); the flexible line 5 is illustrated as connected to an oxygen/pulse sensor 76; and the flexible line 6 is illustrated as connected to a temperature sensor 78.

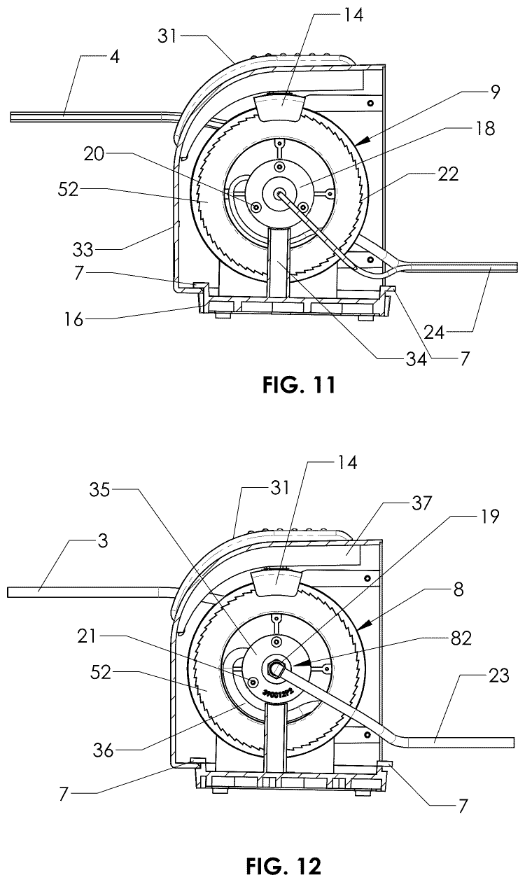

In the illustrated embodiment of the spooled line handling system, the respective flexible line of at least one of said multiple spool assemblies comprises a tube having an interior lumen for fluid passage, with reference at least to the flexible line 3 of the spool assembly 8, which is connected to a blood pressure cuff 80. The flexible line 3 defines an air pressure tube having a lumen for air passage. A rotary air joint 82 (FIG. 11) is connected to the tube opposite the blood pressure cuff 80. The air fitting 36 has a first end coupled to the tube 3 via the rotary air joint 82 and a second end coupled directly or indirectly to a vital signs monitor 2 (FIG. 1).

For each one of the multiple spool assemblies, a non-rotating axle race 30 is affixed to the main frame and retained by the spool. Friction between the spool and the axle race opposes the spring torque thereby damping the rotational speed of the spool at least when the spool rotates in the second rotational direction by the spring torque. Thus the flexible line is returned to the spool by rewinding in a gradual return, preventing damage. In a non-limiting example, the axle race is made of polypropylene plastic. Other durable and smooth materials are within the scope of these descriptions.

Each non-rotating cradle 47 is connected to the mainframe 16 and supports a respective spool assembly. A respective non-rotating clamp 27 is connected at least in part to each cradle 47 by at least one fastener, illustrated as a screw 28 (FIG. 10, 16). The cradle 47 and clamp 27 together define a capture hole 90 (FIG. 10) in which an engagement portion 92 of the axle race 30 is fixedly captured thereby affixing the axle race 30 to the main frame 16. The spool 64 rotates relative to the axle race 30 when the spool is rotated. The engagement portion 92 of the axle race 30 comprises a radially outward facing cylindrical portion 94 and a registration ring 96 (FIG. 17). The cradle 47 and clamp 27 each define a respective radially inward facing arcuate wall (FIG. 16). The arcuate wall 102 of the cradle 47 and the arcuate wall 104 of the clamp 27 together define the capture hole 90 (FIG. 10), and each defines a respective arcuate groove. The arcuate groove 98 of the clamp 27 and the arcuate groove 108 of the cradle 47 together define a registration groove in which the registration ring 96 (FIG. 17) is captured.

Each of the multiple spool assemblies 8, 9, 10, 11 includes a spring, illustrated as a constant force rotary spring 40 (FIG. 17), torsionally coupling the spool 62, 64 to its corresponding axle race 30. The spring provides the spring torque by which the spool to rotates in the second rotational direction 68 (FIG. 3) for retraction of the respective flexible line by rewinding at least a portion thereof onto the spool.

In the illustrated embodiment, each spool 62 (FIG. 19), 64 (FIG. 17) includes a cylinder 100 defining an engagement surface on which the respective flexible line is mounted on the spool in the variably wound condition. A first flange 102 is affixed to a first longitudinal end of the engagement surface of the cylinder 100, and a second flange 104 is affixed to a second longitudinal end of the engagement surface of the cylinder 100. Each flange extends radially outward from the cylinder for maintaining the flexible line on the spool in the variably wound condition. The cylinder of each spool has a center axis 106 coincident with the common axis 60 (FIG. 3) in the assembled condition of the system.

In the illustrated embodiment, each spool 62, 64 includes a ring 110 connected to the first flange 102 and locking teeth 22 extending from an interior of the ring radially inward toward the common axis (106, 60). The locking teeth 22 engage the stop device 70 when the stop device prevents rotation of the spool 62, 64 in the second rotational direction 68. Features of the stop device 70 are described in the preceding descriptions with reference to the spool release plunger 14 with integral ratchet teeth 50. The stop device 70 includes the plunger 14 and attached ratchet teeth 50 (FIG. 15) for engaging the locking teeth 22 of the corresponding spool. The stop device 60 is biased to engage the ratchet teeth 50 with the locking teeth 22.

Features of the release devices 120 (FIG. 16) are described in the preceding descriptions with reference to the illustrated release levers 31. Upon user action on any particular release device 120, the release device moves the stop device 70 thereby disengaging the ratchet teeth 50 from the locking teeth 22. As illustrated each release device comprises the respective pivotable lever 31 having an aperture 46 through which the respective flexible line extends.

In the assembled condition of the system, the cover 33 is attached to the mainframe 16, the mainframe and cover together defining an enclosure in which the multiple spool assemblies 8, 9, 10, 11 are housed. As illustrated, the cover 33 includes, each in one-to-one correspondence with a respective one of the multiple spool assemblies, a lever well 37 at least partially receiving a respective pivotable lever 31. The cover includes a rear panel 17 from which rearward flexible leads extend, each in one-to-one correspondence with a respective one of the multiple spool assemblies, to couple the respective flexible lines to a vital signs monitor 2. Rearward flexible leads refer at least to the non-limiting examples (FIG. 1): blood pressure monitor tube 23; EKG monitor cable 24; oxygen/pulse monitor cable 25; and temperature monitor cable 26.

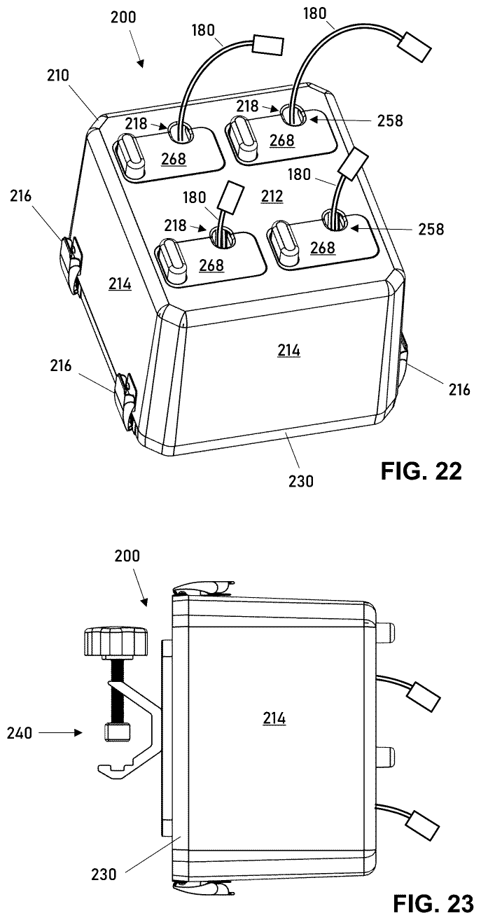

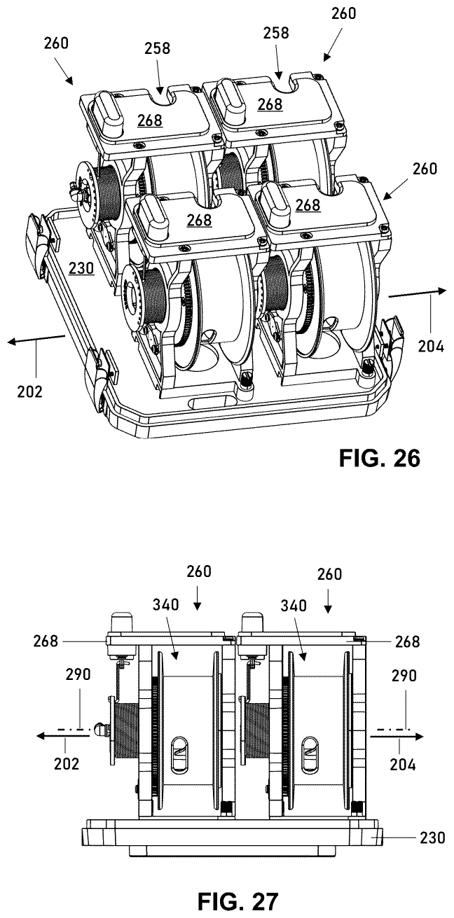

A line management apparatus 200, according to an embodiment shown in FIG. 22, has multiple spool assemblies 260 (FIG. 26), each facilitating use and storage of a respective flexible line 180 (FIG. 22). As with the cable retraction device 1 described in the preceding, the line management apparatus 200 may be utilized to couple vital signs monitor lines such as the blood pressure monitor tube 23, the EKG monitor cable 24, the oxygen/pulse monitor cable 25 and the temperature monitor cable 26 of FIG. 1 to respective flexible lines each with a corresponding device at its deployable end. Use in medical facilities is thus expressly presented. Other uses such as in dental care facilities are anticipated as well. The lines shown or implied extending from the cable retraction device 1 and those shown or implied extending the line management apparatus 200 can carry, convey, contain, collect and/or dispense signals such as electrical and acoustic signals, and flowable substances such as fluids including fluidic gas and fluidic liquid. The subject matters of these descriptions and of the drawings referenced are not limited to uses expressly described or implied herein.

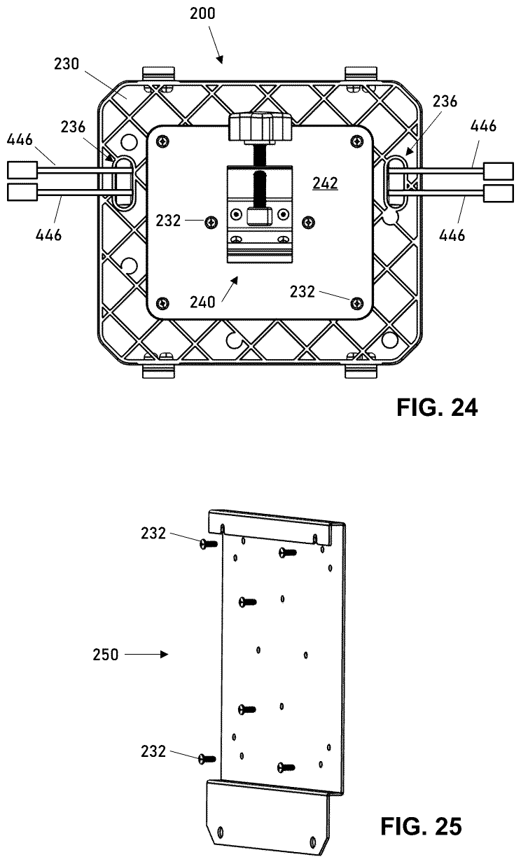

The line management apparatus 200 is shown in FIGS. 22-23 assembled with the cover 210 attached to the base plate 230 by multiple releasable clasps 216. The removable cover has a top side 212, four walls 214, and an open bottom closed out by the rectangular base plate 230 forming an enclosure housing multiple spool assemblies 260 when the cover 210 is in place. The bottom of the base plate 230 shown in FIG. 24 has multiple screw bosses for mounting use, such as to attach the illustrated pole mount bracket 240 (FIG. 24) on the mounting plate 242. A mounting bracket plate 250 is additionally and/or alternatively provided for mounting the line management apparatus 200 to wall, rail, cart or other structure.

The base plate 230 has multiple pass-through slots 236, each for one or more respective flexible line 446 shown extending from the base plate 230 in FIG. 24, representing coupling lines to operatively connect the top-side flexible lines 180 of the line management apparatus 200 to such other devices as a vital signs monitor 2 (FIG. 1), respective fluid source or vacuum lines, electrical lines, sensors and other equipment according to the desired use of the line management apparatus 200 of which no limitation is expressed or implied herein.

Fasteners 232 illustrated as screws are shown in several of the views without exhaustion or cluttering the drawings. Where descriptions herein describe elements as connected without other particular details expressed, it is understood by at least this reference that fasteners such as screws can be used, as a non-limiting example. Where a spool 340 is illustrated and described, a flexible line in a variably wound condition upon the spool 340 is understood or implied with regard to use of the spool 340 without cluttering the drawings.

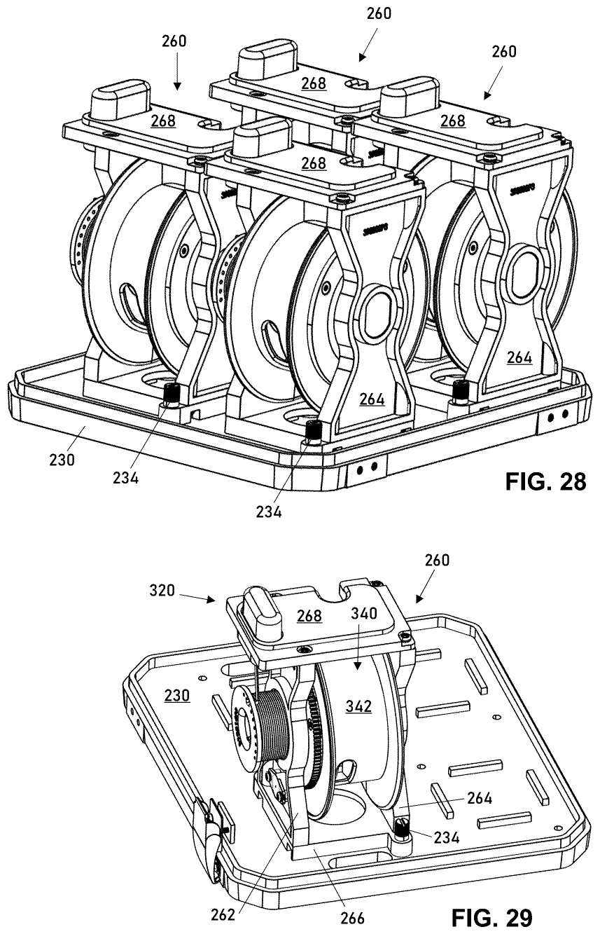

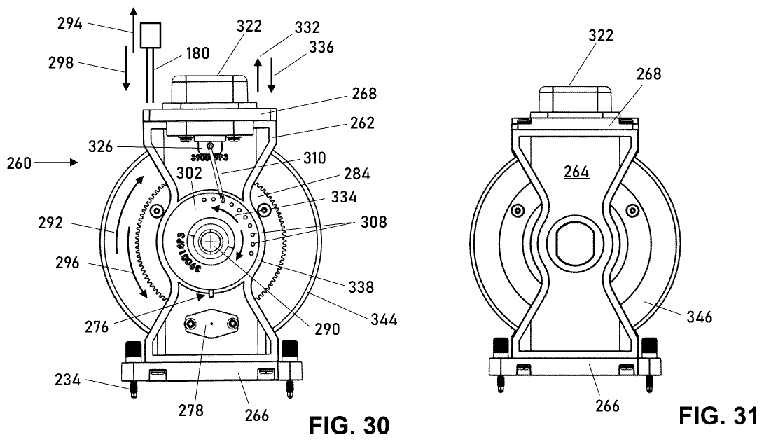

FIG. 26 shows the line management apparatus without the cover for illustration of components. In the following descriptions, the terms front and back refer to features respectively facing or extending in a forward direction 202 and an opposite rearward direction 204, which opposed directions and/or view perspectives are once indicated in FIG. 26 for convenience. These and other relative and directional terms such as side, top and bottom are intuitive for some mountings, implementations and uses of the structures illustrated in FIGS. 22-48. All such terms are used nominally and impart no limitations on absolute directions and orientations of the assembled apparatus in use. For example, the front end of the 260 shown in FIG. 30 can as well be termed the first end, and the opposite back end of spool assembly 260 shown in FIG. 31 can similarly as well be termed the second end opposite the first end. Directional and relative terms used consistently in these descriptions of the structures in FIGS. 22-48 may apply as well to other drawings whether or not such terms are similarly used in descriptions of other drawings. Similarities of the structures of FIGS. 22-48 with those of FIGS. 1-21 are many and apparent, some of which may be signified by similar and same terms in these descriptions, and some of which may not.

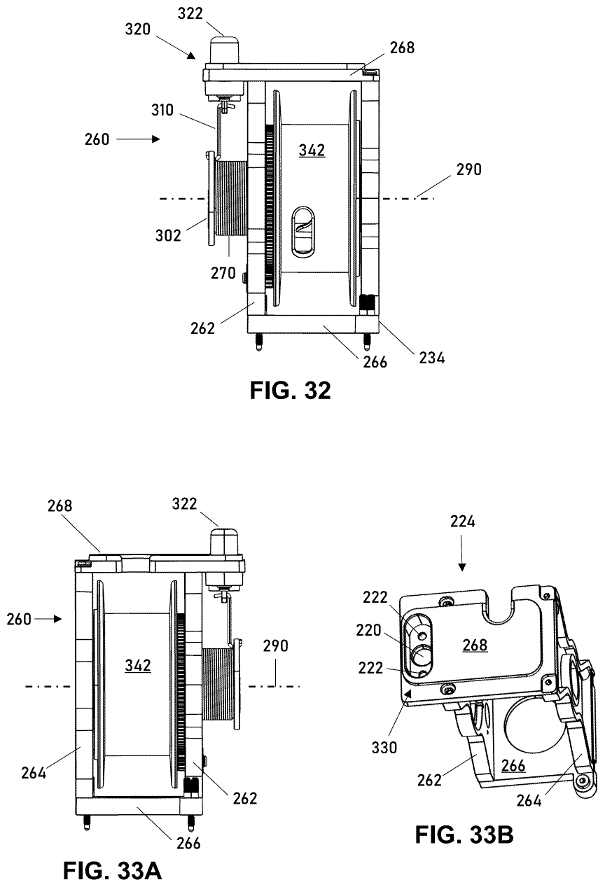

The line management apparatus 200 includes a plurality of spool assemblies 260, each similarly including a rotatable spool 340, and each mounted on the common base plate 230. Each spool 340 is captured between two stanchions, nominally termed a front stanchion 262 and a back stanchion 264 (FIG. 32). The lower end of each stanchion is connected to dedicated mounting plate 266 and each upper end is connected to a dedicated cover plate 268, together forming a four-piece fixed frame 224 (FIG. 33B) dedicated to the particular spool assembly 260.

Each cover plate 268 has a notch 258 that aligns with a respective corresponding notch 218 of the cover 210 (FIG. 22), which together form a pass-through slot through which a respective flexible line 180 can extend and withdraw independently of the others.

Advantageously, each fixed frame 224 is mounted independently to the base plate 230 (FIG. 29) by fasteners 234 through mounting holes at opposing corners of the rectangular mounting plate permitting independent removal and servicing of each spool assembly 260. The following descriptions of the singly illustrated spool assembly 260 in FIGS. 29-44 are sufficient with regard to the four spool assemblies 260 in FIGS. 26-28.

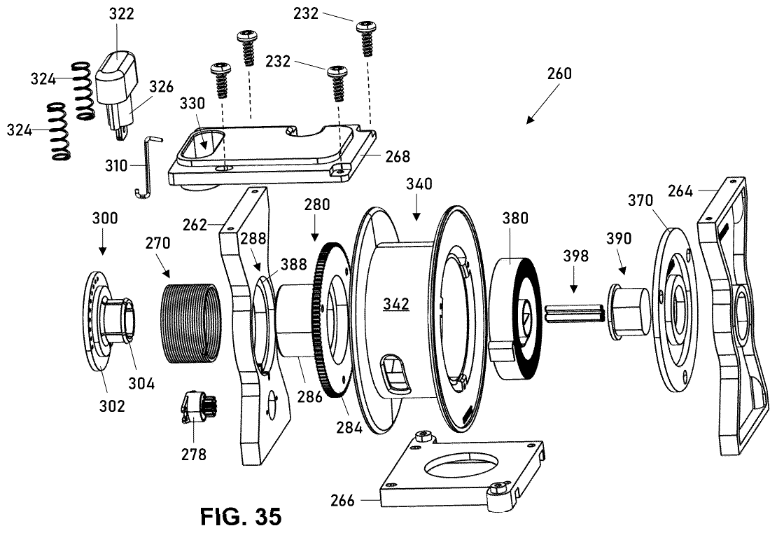

The spool assembly 260 includes a rotating hub 280 (FIG. 35, 37). The hub 280 is connected to and extends forward from the front end of the spool 340. The hub 280 rotates with the spool 340. A smooth cylindrical forward portion of the hub 280, termed herein a hub axle 282, extends forward through a central through-hole 288 of the front stanchion 262. The hub axle 282 rotates smoothly in the through hole 288 and with the spool 340. The smooth interior wall of the through hole 288 serves as a race and the hub axle 282 serves as a rotation axle for the front end of the spool 340. A spur gear 284 of the hub 280 is integrally connected to the back end of the hub axle 282. The spur gear 284 is defined by an annular flange and gear teeth along the circular outer perimeter of the flange.

The forward direction 202 and rearward direction 204 are chosen for convenience in these descriptions as opposing directions along or with respect to a rotational axis 290, shown for reference in FIGS. 26-27. The spool 340 of each spool assembly 260 operatively rotates around its rotational axis 290. In the illustrated embodiment of the line management apparatus 200, four spool assemblies 260 are arranged in a rectangular two row by two column arrangement in which the rotational axis 290 or each spool assembly 260 is common with another.

The spool 340, and components that rotate therewith, rotate around the rotational axis 290 in a first rotational direction 292 (FIG. 30) corresponding to unwinding a flexible line 180 from the spool 340 and extending the line from the line management apparatus 200 for deployment and use of the line as represented by the extend direction 294. The spool 340 and components that rotate therewith also rotate in a second rotational direction 296, which is opposite the first rotational direction 292, corresponding to rewinding the flexible line 180 onto the spool 340 thereby withdrawing the line into the line management apparatus 200 as represented by the withdraw direction 298 opposite the extend direction 294 in FIG. 30. The first rotational direction 292 and second rotational direction 296 are accordingly also termed respectively the unwind direction 292 and rewind direction 296 for convenience in the following descriptions.

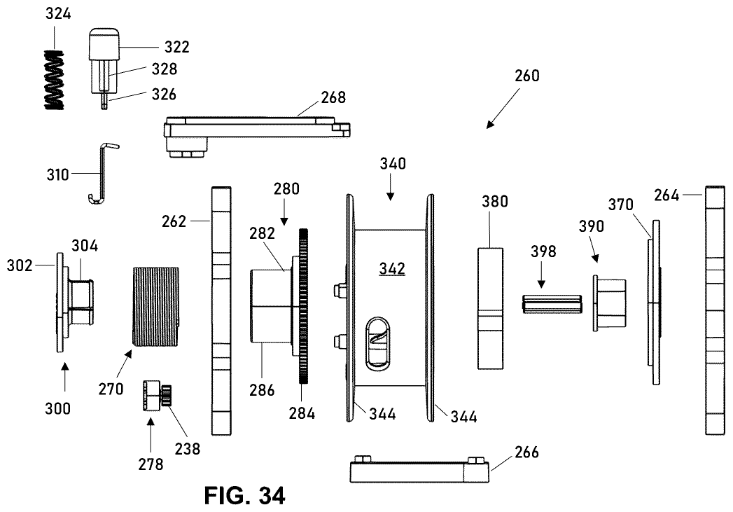

A rotary damper 278 operatively coupled to the spool 340 is illustrated as connected to the lower end of the front stanchion has a spur gear 238 (FIG. 34) engaging the spur gear 284 of the hub and damping rotation of the hub and spool in the rewind direction. This feature advantageously facilitates a safe and orderly return of an extended line under bias by a biasing element of which the recoil spring 380 is an illustrated embodiment.

A smooth cylindrical clutch engagement portion 286 of the hub axle 282 extends forward through the front stanchion 262 and is variably engaged by a stop device, which includes a clutch element 270 at least partially surrounding the hub axle 282, and a rotationally biased clutch-release cap 300. The clutch element is a torsion spring mounted around the clutch engagement portion 286 and rotational axis 290 in the illustrated embodiment. The clutch element 270 is coupled to both the front stanchion and to the clutch-release cap 300. The rotational position of to the clutch-release cap 300 effectively controls engagement of the clutch element 270 with the hub axle 286. In an engaged state, the clutch element 270 permits rotation of the hub 280 in the unwind direction 292, but prevents rotation thereof in the rewind direction 296 by frictional engagement with the clutch engagement portion 286 of the hub axle 282. In a disengaged state, the clutch element 270 permits rotation of the hub 280 in any rotational direction, including the rewind direction 296 with rotational speed damping applied by the rotary damper 278.

The clutch-release cap 300 includes a front flange 302 and an annular clip 304 (FIG. 34-35, 38) integrally connected thereto and extending rearward therefrom. The rotational position of the clutch-release cap 300 is controlled by rigid link member 310 (FIG. 30) having a lower end pivotally coupled to the front flange 302 and an upper end pivotally coupled to a linearly biased release device 320. The lower end and upper end of the link member 310 are each illustrated as a hook (FIG. 34).

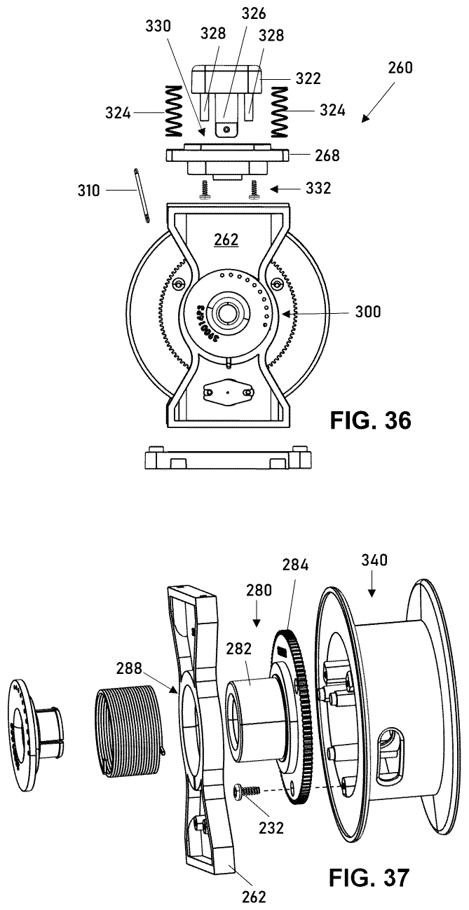

The release device 320 is carried by the cover plate 268 and includes a plunger 322 (FIG. 36) biased away from the clutch-release cap 300 by two coil springs 324. The top of the plunger 322 serves as a button extending outward from the cover plate 268 for user force downward to overcome the upward bias force applied by the coil springs 324. The plunger 322 has a tab 326 extending downward from the button and variably through a hole 220 in the floor of a well 330 (FIG. 33B) defined by the cover plate 268. The plunger 322 (FIG. 34-36) includes a spaced pair of posts 328 flanking the tab 326 and passing variably through respective post holes 222 through the floor of the well 330 flanking the hole 220 through which the tab passes. The springs 324 are trapped on the posts between the bottom of the button and floor of the well 330. A respective fastener, illustrated as a screw 232 (FIG. 36) with a head too large to pass through the post hole 222, engages the bottom end of each post 328, the fasteners together maintaining assembly of the plunger 322 and springs 324 with the cover plate 268. The tab 326 thus persistently biases the link member 310 away from the clutch-release cap 300 and toward the cover plate 268.

The sloped direction of the link member 310 from the tab 326 to the front flange 302 and thus the vector force of the link member 310 applied to the clutch-release cap 300 are non-central with regard to the rotational axis 290, and thus the link member 310 applies a torque upon the clutch-release cap 300 biasing the rotational position of the clutch-release cap 300 which biases the clutch element 270 to its engaged state. That is, linear upward movement 332 (FIG. 30) of the plunger 322 by the force of the springs toward its top position relative to top cover plate 268 rotates the front flange 302 in one rotational direction 334, engaging the clutch element 270. Linear downward movement 336 of the plunger 322 by user action overcoming the force of the springs rotates the front flange 302 in a rotational direction 338 opposite the one rotational direction 334, disengaging the clutch element 270.

The rotational position range of travel of the clutch-release cap 300 upon movement of the plunger 322 is preset by the position at which the lower end of the link member 310 is pivotally coupled to the front flange 302. The front flange includes multiple angularly spaced engagement holes 308 for selection of use by which to engage the link member 310 and clutch-release cap 300 by hooking the lower of the link member 310 to a selected engagement hole 308, thus varying the clutch engagement and allowing clutch action adjustment of the clutch element.

The illustrated one rotational direction 334 of the front flange corresponding to clutch engagement and the rewind rotational direction 296 corresponding to rotation of the spool 340 are both shown as counterclockwise in FIG. 30. This is due to particular arrangements of the illustrated spool assembly 260 and the torsion spring that defines the clutch element 270 in the illustrated embodiment. Other embodiments otherwise within the scope of these descriptions may vary in such arrangements.

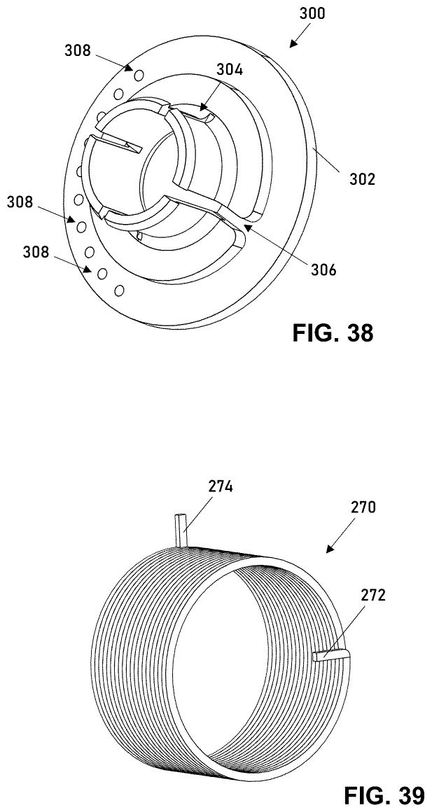

The torsion spring (clutch element 270) is operatively coupled to both the front stanchion 262 and to the clutch-release cap 300 so as to vary its wound condition around and upon the clutch engagement portion 286 of the hub 280 as the plunger and link 310 cause pivoting of the clutch-release cap 300. The front end of the torsion spring has a radially inward extending prong 272 (FIG. 39) received by a radial slot 306 defined by the annular clip (FIG. 38). The back end of the torsion spring has a radially outward extending prong 274 received by a radial slot 276 defined by the front stanchion 262 (FIG. 30). The back end of the torsion spring is thus irrotationally engaged with the fixed frame 224 by way of the front stanchion 262 and the fixed frame 224. The front end of the torsion spring 270 pivots with the clutch-release cap 300 as the user acts upon the plunger 322, loosening the wound condition of the torsion spring 270 and effectively disengaging the torsion spring from the hub 280 when the top of the plunger 322 is pressed like a button permitting the clutch engagement portion 286 of the hub axle 282 to rotate within the torsion spring.

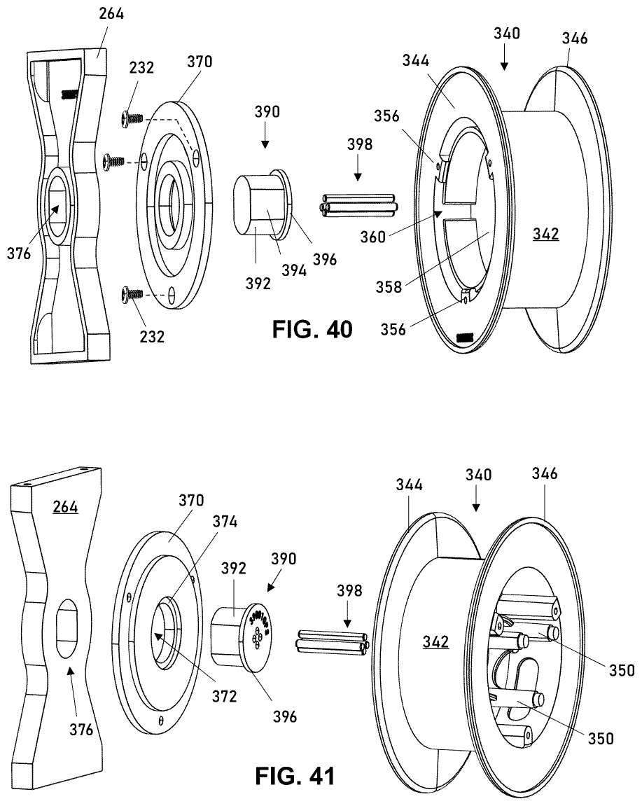

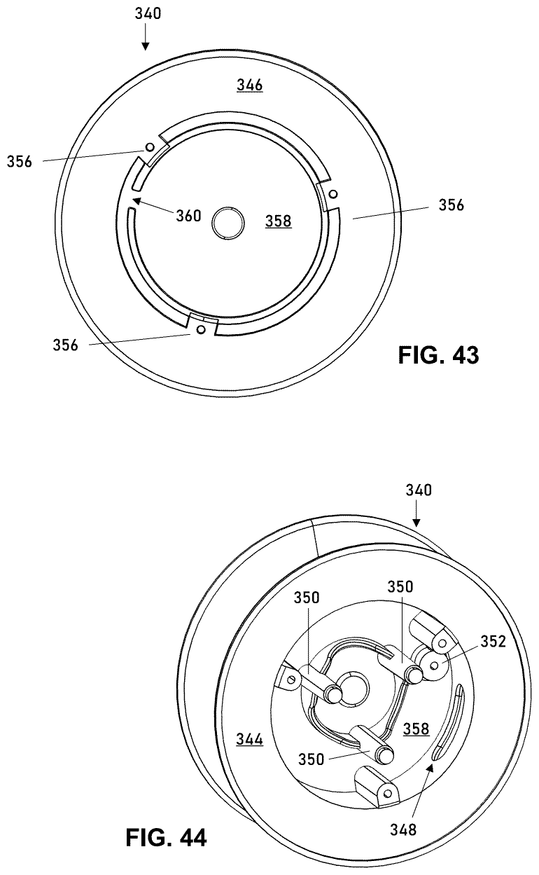

At the rear of the spool 340, the spool assembly 260 includes a rotating annular retainer 370 (FIGS. 34-35) trapping a recoil spring 380 within the spool 340 and in engagement with the spool 340. The retainer 370 is connected to the back end of the spool 340 and rotates with the spool 340. An integral annular divider wall 358 (FIG. 43) perpendicular to the rotation axis divides the interior of the spool 340 into a front chamber facing the hub 280 and a back chamber in which the recoil spring 380 is trapped by the annular retainer. Supports 356 integrally connected to the interior wall of the cylinder 342 rearward of the annular divider wall 358 receive fasteners, such as screws 232 in FIG. 40, to connect the retainer 370 to the back end of the spool 340.

An irrotational axle cap 390 (FIG. 40) has an axle portion 392 that extends back through the retainer 370 and into a central through-hole 376 of the back stanchion 264. The axle portion 392 of the axle cap 390 has cylindrical sections separated by secant facets 394 that irrotationally register with corresponding secant facets of the through-hole 376. The retainer 370 has a central through hole 372 through which the axle portion extends. The smooth interior of the through hole 372 serves as a race for the axle portion permitting the spool 340 and retainer 370 to rotate freely on the axle portion. The front of the irrotational axle cap 390 has a lip 396 received by an annular ledge 374 (FIG. 41) around the through hole 372 at the front of the retainer 370 facing the spool 340. The axle cap 390 is captured by the retainer 370 when the retainer 370 is connected to the spool 340, for example by fasteners illustrated as screws 232 in FIG. 40.

The spool 340 is accordingly rotatable within the fixed frame defined by the mounting plate 266, front stanchion 262, back stanchion 264, and cover plate 268. At the front of the spool 340, the forward extending hub axle 282 serves as an axle for the spool turning within the through hole 288 of the front stanchion 262. At the back of the spool 340, the retainer 370 turns around the irrotational axle portion 392 of the axle cap 390 supported by the back stanchion.

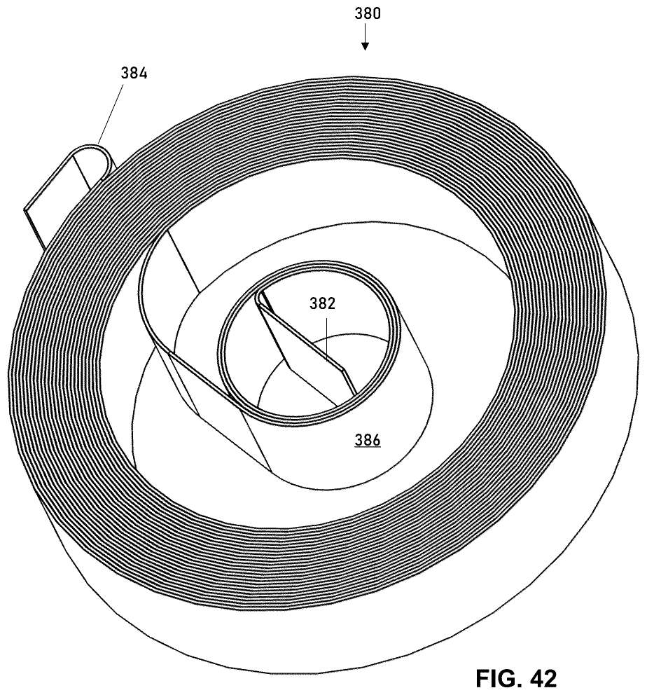

An irrotational spring stay 398 (FIGS. 40-41) is mounted on the axle cap 390 and thus is connected irrotationally to the fixed frame via the axle cap 390 connection to the back stanchion 264. The spring stay 398 extends forward from the front of the axle cap 390 to engage an interior end portion 382 of the recoil spring 380 (FIG. 42), which is illustrated as a spiral coil spring. A rotating slot 360 (FIG. 40) integral with the interior of the spool 340 engages an exterior hooked end portion 384 of the recoil spring 380. The spool 340 is rotationally biased by a torque provided by the recoil spring 380 perpetually in the rewind direction 296 and against the unwind direction 292. As the spool 340 is rotated in the unwind direction 292 (FIG. 30), for example by manual force upon a flexible line 180 being extended for use, an increasing tightly wound portion 386 of the interior of the recoil spring 380 accumulates around the irrotational spring stay 398, tightening the recoil spring 380 as the slot 360 and hooked end portion of the recoil spring 380 rotate.

Upon disengagement of the clutch element 270, the recoil spring 380 motivates the spool 340 to return in the rewind direction with rotational speed damping applied by the rotary damper 278, safely and orderly rewinding the flexible line 180 onto the spool. The spring stay 398 is illustrated as pins, such as dowels or rolls each pressed into a respective bore in the front end of the axle cap 390. The pins are arranged parallel to and spaced around the rotational axis. The interior end portion 382 of the recoil spring 380 is irrotationally captured between forward portions of the pins extending forward from the axle cap 390.

Each spool assembly 260 in a utilized implementation carries a deployable flexible line referenced as line 180 in several drawings. The flexible line can be of any utility a user prefers. In some embodiments and uses, such a flexible line may be for fluid handling. Fluid handling refers to dispensing by pressure, and collecting by vacuum, fluidic gas, fluidic liquid, combinations thereof, and entrained particulate matter or substances without limitation.

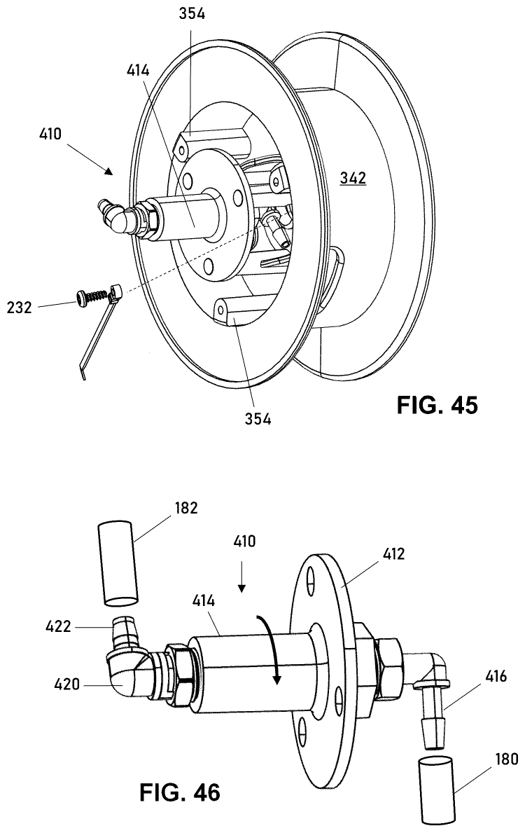

FIG. 45 is a perspective view of the front end of the spool 340 of FIGS. 43 and 44, shown partially assembled with a rotary fluid connection device 410. The fluid connection device includes an annular plate 412 connected to the front end of the spool 340, and a conduit pipe 414 integrally connected to the annular plate and extending forward therefrom around the rotation axis. An internal swaged fitting tube 416 is mechanically connected to the annular plate 412 and fluidically interiorly open to the conduit pipe 414 for passage of fluid therethrough to a swivel coupler 420 having an external swaged fitting tube 422. The swivel coupler 420 and external swaged fitting tube 422 thereof permit free rotation of the remainder of the rotary fluid connection device 410, including the annular plate 412, the conduit pipe 412 and the internal swaged fitting tube 416, which all rotate with the spool 340.

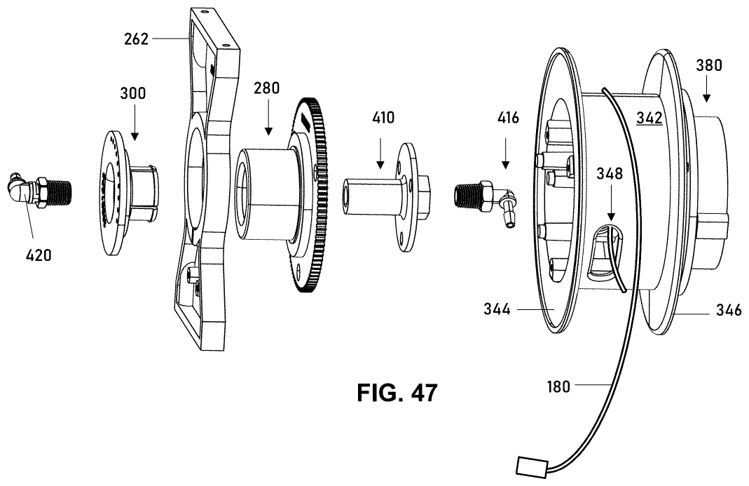

The front spool-external swaged fitting tube 422 facilitates external fluid connection to a fluid pressure or vacuum source 182, as representing in FIG. 46, without the source winding about any rotating portion of the spool assembly 260. The spool-internal swaged fitting tube 416 facilitates internal fluid connection to the terminal end of a flexible line 180, illustrated in FIG. 46, the remainder of which can wind and unwind from the spool 340 as represented in FIG. 47. The illustration of the flexible line 180 shown in FIG. 47 is not intended as necessarily scale with regard to its length, which in typical use may wrap several times around the spool.

The annular plate 412 of the fluid connection device 410 is captured between the spool 340 and hub 280 and rotates therewith. Supports 354 (FIG. 47) integrally connected to the interior wall of the cylinder 342 forward of the annular divider wall 358 receive fasteners, singly represented as a screw 232 in FIG. 37, by which the hub 280 is secured to the front end of the spool 340 by way of the spur gear 248 having holes aligned with the supports upon assembly.

Mounting pedestals 350 (FIG. 44) with diametrically reduced termini are integral with the interior of the spool 340 and extend forward therein. Corresponding mounting holes of the annular plate 412 receive the termini registering the annular plate 412 and spool 340 to rotate together. The outer circular edge of the annular plate is nested by an interior annular ledge 388 (FIG. 35) within the hub 280 between the spur gear 284 and hollow hub axle 282 (FIG. 34), trapping the annular plate 412 on the termini of the mounting pedestals 350.

In the illustrated embodiment, the cylinder 342 of the spool 340 defines an engagement surface on which the respective flexible line is mounted on the spool 340 in a variably wound condition. A front flange 344 is integrally connected to the front end of the cylinder 342, and a back flange 346 is integrally connected to the back end of the cylinder spaced from the front flange 344 by the engagement surface. Each spool flange extends radially outward from the cylinder 342 for maintaining the flexible line 180 on the spool 340 in the variably wound condition. The cylinder 342 has a center axis coincident with the rotation axis. A circumferentially extending slot 348 (FIG. 45) through the wall of the cylinder forward of the annular divider wall 358 accommodates passage of the terminal end of a flexible line 180 through the cylinder for coupling to the internal swaged fitting tube. A fastener 184 secures the end of the flexible line 180 within the spool 340 to support any tension applied without freeing the terminal end from the internal swaged fitting tube 416. The fastener 184 illustrated in FIG. 45 is a mountable cable tie with an eyelet that receives a screw 232 that attaches the cable tie, and the flexible line 180 therewith when the cable tie captures the line, to a boss 352 integral with the interior of the spool 340 (FIG. 44).

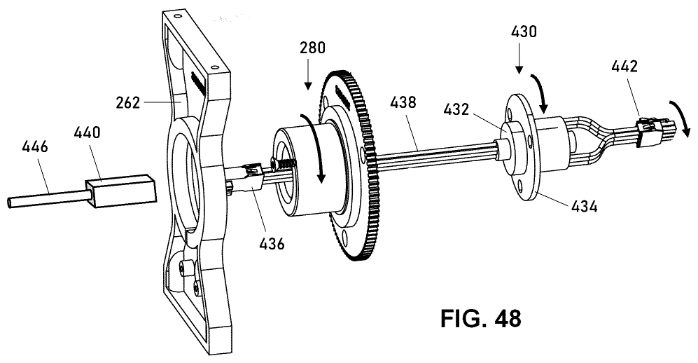

FIG. 48 is a perspective view of the front stanchion, hub 280, and a rotary electrical connection device 430 for use with the spool assembly 260 of FIG. 22. The electrical connection device 430 includes a slip ring 432 rotatable relative to an annular plate 434 that connects to the front end of the spool 340 similarly as with the annular plate 412 of the fluid connection device 410. The annular plate 434 of the electrical connection device 412 is trapped by the interior annular ledge 388 (FIG. 35) within the hub 280 between the spur gear 284 and hollow hub axle 282, trapping the annular plate 434 on the termini of the mounting pedestals 350 when installed. A spool-external wiring harness connector 436 extends forward through the hub 280 and clutch-release cap 300 on a forward multi-wire cable 438 for coupling to non-rotating mating harness connector or device 440. A spool-internal wiring harness 442 extends rearward from the slip ring into the interior of the spool 340 to facilitate internal electrical connection to a flexible electrical line extending through the slot 348 of the cylinder 342. The slip ring 432 permits free rotation of internal wiring harness 442 relative to the external wiring harness as a flexible electrical line 180 is wound and unwound from the spool 340. A flexible line 446, to which the connector or device 440 is connected in FIG. 48, is shown extending from the base plate 230 in FIG. 24, representing coupling lines to operatively connect the flexible lines 180 of the line management apparatus 200 to such other devices as a vital signs monitor 2 (FIG. 1), respective fluid source or vacuum lines, electrical lines, sensors and other equipment according to the desired use of the line management apparatus 200 of which no limitation is expressed or implied herein.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

What is claimed is:

1. A spool assembly comprising:
a spool having a rotation axis;
a fixed frame on which the spool is mounted;
a biasing element operatively coupled to the spool to apply a torque against rotation of the spool in a first rotational direction around the rotation axis relative to the fixed frame;
a flexible line mounted on the spool in a variably wound condition such that, when the flexible line is extended by unwinding from the spool, the spool is rotated in the first rotational direction against the torque provided by the biasing element;
a stop device coupled to the spool selectively preventing rotation of the spool in a second rotational direction opposite the first rotational direction thereby selectively preventing, by opposing at least the torque provided by the biasing element, retraction of the flexible line by winding onto the spool;
a release device operatively coupled to the stop device such that, upon action on the release device, the release device selectively releases the stop device to allow the spool to rotate in the second rotational direction by the torque provided by the biasing element thereby selectively permitting retraction of the flexible line by rewinding at least a portion thereof onto the spool; and
a rotary damper operatively coupled to the spool to damp rotation of the spool at least in the second rotational direction.

2. The spool assembly of claim 1, further comprising a hub connected to the spool to rotate therewith, the hub comprising a smooth cylindrical axle, wherein:
the stop device comprises a clutch element at least partially surrounding a smooth cylindrical clutch engagement portion of the axle, the clutch element having an engaged condition and a disengaged condition; and
in the engaged condition, the clutch element selectively prevents rotation of the axle and spool therewith in the second rotational direction by frictional engagement with the smooth cylindrical clutch engagement portion of the axle.

3. The spool assembly of claim 2, wherein the hub comprising a spur gear engaged by the rotary damper.

4. The spool assembly of claim 2, wherein the clutch element comprises a torsion spring mounted around the smooth cylindrical clutch engagement portion of the axle, and in the engaged condition of the clutch element the torsion spring prevents rotation of the axle and spool therewith in the second rotational direction by frictional engagement with the clutch engagement portion of the axle.

5. The spool assembly of claim 4, wherein the release device is operatively coupled to the torsion spring to vary a wound condition of the torsion spring around and upon the clutch engagement portion of the axle.

6. The spool assembly of claim 5, wherein:
a first end of the torsion spring is operatively coupled to the release device; and
a second end of the torsion spring opposite the first end is irrotationally engaged with the fixed frame.

7. The spool assembly of claim 6, wherein the release device comprises a clutch release cap engaged with the first end of the torsion spring such that upon a pivoting of the clutch release cap around the rotation axis, the wound condition of the torsion spring around and upon the clutch engagement portion of the axle is loosened.

8. The spool assembly of claim 7, wherein the release device comprises a plunger mounted on the fixed frame and a link member operatively coupling the plunger to the clutch release cap such that linear movement of the plunger causes pivoting of the clutch release cap around the rotation axis.

9. The spool assembly of claim 8, wherein the clutch release cap comprises a flange having multiple spaced engagement features for selection of use by which to engage the link member and clutch-release cap for adjustment of the clutch element.

10. The spool assembly of claim 1, wherein:
the biasing element comprises a recoil spring within the spool;
a spring stay is connected irrotationally to the fixed frame;
an interior end portion of the recoil spring engages the spring stay and is prevented from rotating relative to the fixed frame by the spring stay; and
an exterior hooked end portion of the recoil spring engages an interior feature of the spool such that the recoil spring applies said torque against rotation of the spool in the first rotational direction.

11. The spool assembly of claim 8, wherein the recoil spring is configured such that, when the flexible line is extended by unwinding from the spool, the spool is rotated in the first rotational direction and an increasing tightly wound interior portion of the recoil spring is accumulated around the spring stay, tightening the recoil spring.

12. The spool assembly of claim 1, wherein the fixed frame comprises
a mounting plate;
a first stanchion mounted on the mounting plate;
a second stanchion mounted on the mounting plate and spaced from the first stanchion; and
a cover plate mounted on the first stanchion and second stanchion and spaced from the mounting plate,
wherein the spool is mounted on the frame between the first stanchion and second stanchion, and wherein the spool is mounted on the frame between the mounting plate and cover plate.

13. The spool assembly according to claim 1, wherein the spool further comprises:
a cylinder defining an engagement surface on which the flexible line is mounted on the spool in the variably wound condition;
a first flange connected to the cylinder and extending radially outward from the cylinder for maintaining the flexible line on the spool in the variably wound condition; and
a second flange connected to the cylinder, spaced from the first flange by the engagement surface, and extending radially outward from the cylinder for maintaining the flexible line on the spool in the variably wound condition.

14. The spool assembly according to claim 13, wherein the spool further comprises a slot through the cylinder and through which a terminal end of a flexible line passes through the cylinder.

15. The spool assembly according to claim 14, wherein the flexible line is configured to at least one of carry, convey, contain, collect and dispense at least one of a signal and a flowable substance.

16. The spooled line handling system according to claim 15, further comprising a cover connected directly or indirectly to the fixed frame and at least partially defining an enclosure around the spool assembly.
